(19) United States
(12) Patent Application Publication
(10) Pub. No.: US 2017/0304313 A1
(43) Pub. Date: Oct. 26, 2017

(54) Therapeutic Combination For The Treatment Of Cancer

(71) Applicant: Novartis AG, Basel (CH)

(72) Inventors: Dale Porter, Cambridge, MA (US); Nafeeza Hafeez, Cambridge, MA (US)

(21) Appl. No.: 15/517,035

(22) PCT Filed: Oct. 2, 2015
(86) PCT No.: PCT/IB2015/057550

§ 371 Date: Apr. 5, 2017

Related U.S. Application Data
(60) Provisional application No. 62/060,066, filed on Oct. 6, 2014.

(51) Int. Cl. A61K 31/55; A61K 31/496; A61K 31/506; A61K 45/06

(52) U.S. Cl. CPC A61K 31/55; A61K 45/06; A61K 31/496; A61K 31/506

(57) ABSTRACT

This invention relates to a pharmaceutical combination comprising (a) an EGFR inhibitor and (b) a FGFR inhibitor, particularly for use in the treatment of a cancer. This invention also relates to uses of such combination for preparation of a medicament for the treatment of a cancer; methods of treating or preventing a cancer in a subject in need thereof comprising administering to said subject a jointly therapeutically effective amount of said combination; pharmaceutical compositions comprising such combination and commercial packages thereto.

### TECHNICAL FIELD

This invention relates to a pharmaceutical combination comprising (a) an EGFR inhibitor and (b) a FGFR inhibitor, particularly for use in the treatment of a cancer. This invention also relates to uses of such combination for preparation of a medicament for the treatment of a cancer; methods of treating or preventing a cancer in a subject in need thereof comprising administering to said subject a jointly therapeutically effective amount of said combination; pharmaceutical compositions comprising such combination and commercial packages thereto.

### BACKGROUND ART

The epidermal growth factor receptor (EGFR, Erb-B1) belongs to a family of proteins involved in the proliferation of normal and malignant cells. Overexpression of EGFR is found in over 70 percent of human cancers, including without limitation non-small cell lung carcinomas (NSCLC), breast cancers, gliomas, squamous cell carcinoma of the head and neck, and prostate cancer. The identification of EGFR as an oncogene has led to the development of EGFR tyrosine kinase inhibitors, such as gefitinib, erlotinib, or afatinib, and their deployment in a treatment of cancers, in particular non-small cell lung cancer, harboring EGFR mutations. However, almost all patients develop with the time resistance to the treatment with such EGFR tyrosine kinase inhibitors as gefitinib, erlotinib, or afatinib.

Therefore, in spite of numerous treatment options for patients suffering from a cancer, in particular NSCLC, there remains a need for effective therapeutics preventing or delaying development of resistance in the course of treatment with EGFR tyrosine kinase inhibitors; or overcoming or reversing resistance acquired in the course of treatment with EGFR tyrosine kinase inhibitors.

### SUMMARY OF THE INVENTION

It is an object of the present invention to provide for a medicament to improve treatment of a cancer, particularly lung cancer, particularly cancer characterized by mutant EGFR, and particularly cancer with acquired resistance to a treatment with an EGFR tyrosine kinase inhibitor, or developing a resistance to a treatment with an EGFR tyrosine kinase inhibitor, or under high risk of developing a resistance to a treatment with an EGFR tyrosine kinase inhibitor.

In accordance with the present invention, it has been found that a combination of an EGFR inhibitor, selected from the novel group of highly potent EGFR inhibitors, with specific FGFR inhibitors has a beneficial synergistic interaction, improved anti-cancer activity, improved anti-proliferative effect, and improved durability of the response, e.g., with regard to the delay of progression or inhibiting a cancer or its symptoms, particularly in cancers characterized by mutant EGFR, and particularly cancer with acquired resistance to a treatment with an EGFR tyrosine kinase inhibitor, or developing a resistance to a treatment with an EGFR tyrosine kinase inhibitor, or under high risk of developing a resistance to a treatment with an EGFR tyrosine kinase inhibitor.

In one aspect, the present invention relates to a pharmaceutical combination, referred to as a COMBINATION OF THE INVENTION, comprising (a) a compound of formula I

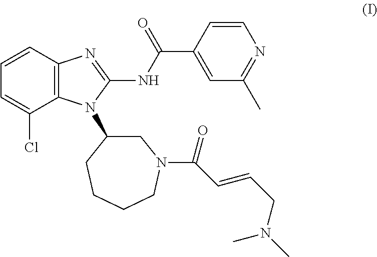 [SMILES: Cc1cc(C(=O)Nc2nc3cccc(Cl)c3n2[C@@H]2CCCCN(C(=O)/C=C/CN(C)C)C2)ccn1]

(R,E)-N-(7-chloro-1-(1-(4-(dimethylamino)but-2-enoyl)azepan-3-yl)-1H-benzo[d]imidazol-2-yl)-2-methylisonicotinamide, or a pharmaceutically acceptable salt thereof, and (b) a FGFR inhibitor.

In another aspect, the present invention relates to the COMBINATION OF THE INVENTION for simultaneous, separate or sequential use.

In another aspect, the present invention relates to the COMBINATION OF THE INVENTION for use in the treatment of a cancer, particularly lung cancer.

In one aspect, the present invention relates to a method of treating a cancer comprising simultaneously, separately or sequentially administering to a subject in need thereof the COMBINATION OF THE INVENTION in a quantity which is jointly therapeutically effective against said lung cancer.

### DETAILED DESCRIPTION OF THE FIGURES

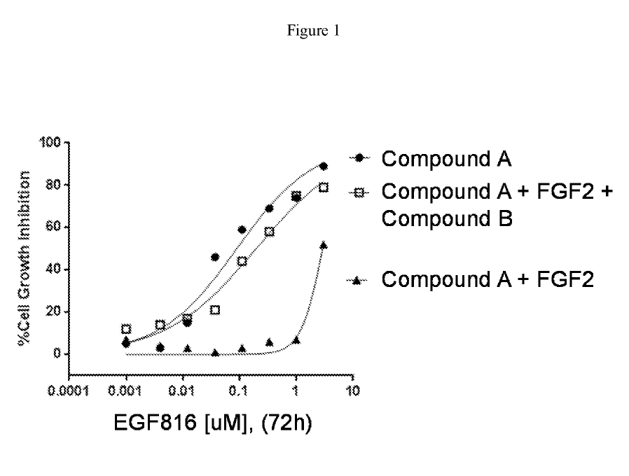

FIG. 1: Percent cell growth inhibition of the NCI-H1975 EGFR mutant cell line following 72 hours of treatment with several doses of (R,E)-N-(7-chloro-1-(1-(4-(dimethylamino)but-2-enoyl)azepan-3-yl)-1H-benzo[d]imidazol-2-yl)-2-methylisonicotinamide (herein referred to as “COMPOUND A”) as a single agent, plus FGF2, or plus both FGF2 and 3-(2,6-Dichloro-3,5-dimethoxy-phenyl)-1-{6-[4-(4-ethyl-piperazin-1-yl)-phenylamino]-pyrimid-4-yl}-1-methyl-urea (500 nM) (herein referred to as “COMPOUND B”).

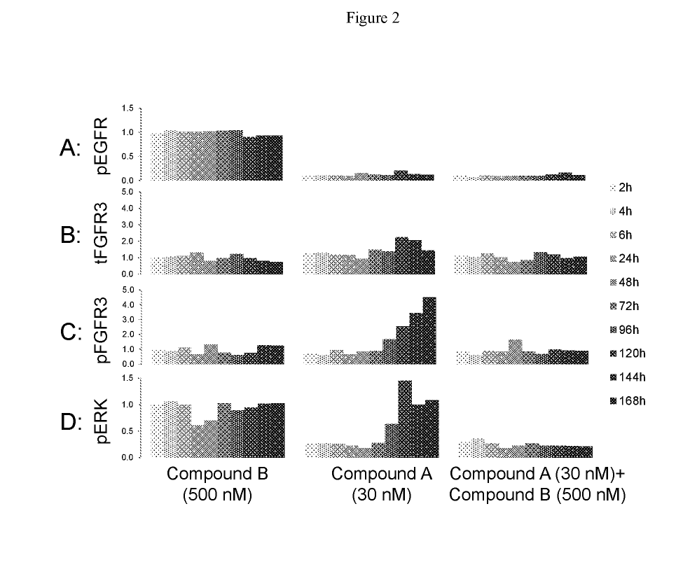

FIG. 2: Fold-changes in pEGFR (A), tFGFR3 (B), pFGFR3 (C), and pERK (D) following treatment of the EGFR mutant NCI-H1975 cell line with COMPOUND B (500 nM) alone, COMPOUND A (30 nM) alone, or the combination of COMPOUND A (30 nM) and COMPOUND B (500 nM), are shown relative to vehicle treated control over a time-course. All Y-axes represent fold-change versus vehicle.

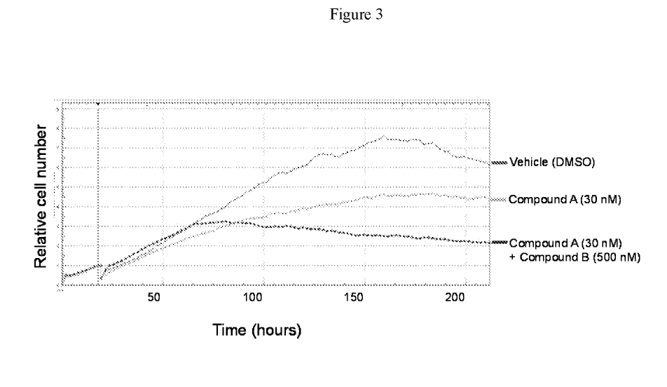

FIG. 3: Relative cell number following treatment of the EGFR mutant NCI-H1975 cell line with Vehicle (DMSO), COMPOUND A (30 nM) alone, or the combination of COMPOUND A (30 nM) plus COMPOUND B (500 nM) over time.

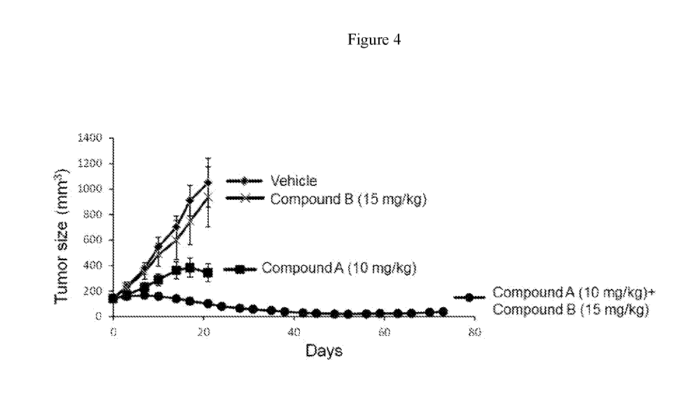

FIG. 4: Tumor size of patient derived primary tumor xenografts bearing EGFR mutations (L858R; T790M) grown in mice. The mice were treated with Vehicle, COMPOUND A (10 mg/kg/day) as a single agent, COMPOUND B (15 mg/kg/day) as a single agent; or the combination of COMPOUND A (10 mg/kg/day) plus COMPOUND B (15 mg/kg/day).

### DETAILED DESCRIPTION OF THE INVENTION

In one aspect, the present invention relates to a pharmaceutical combination comprising (a) a compound of formula I

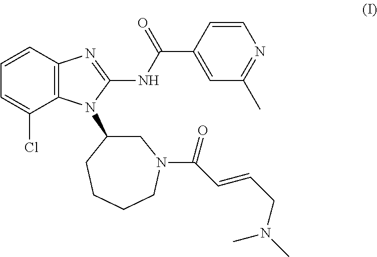 [SMILES: Cc1cc(C(=O)Nc2nc3cccc(Cl)c3n2[C@@H]2CCCCN(C(=O)/C=C/CN(C)C)C2)ccn1]

(R,E)-N-(7-chloro-1-(1-(4-(dimethylamino)but-2-enoyl)azepan-3-yl)-1H-benzo[d]imidazol-2-yl)-2-methylisonicotinamide (herein referred to as “COMPOUND A”), or a pharmaceutically acceptable salt thereof, and (b) a FGFR inhibitor.

The term “combination” or “pharmaceutical combination” is defined herein to refer to either a fixed combination in one dosage unit form, a non-fixed combination or a kit of parts for the combined administration where the therapeutic agents, e.g., the compound of formula I and the FGFR inhibitor, may be administered together, independently at the same time or separately within time intervals, which preferably allows that the combination partners show a cooperative, e.g. synergistic effect.

The term “fixed combination” means that the therapeutic agents, e.g., the compound of formula I and the FGFR inhibitor, are in the form of a single entity or dosage form.

The term “non-fixed combination” means that the therapeutic agents, e.g., the compound of formula I and the FGFR inhibitor, are administered to a patient as separate entities or dosage forms either simultaneously, concurrently or sequentially with no specific time limits, wherein preferably such administration provides therapeutically effective levels of the two therapeutic agents in the body of the subject, e.g., a mammal or human in need thereof.

The term “synergistic effect” as used herein refers to action of two therapeutic agents such as, for example, (a) the compound of formula I, and (b) a FGFR inhibitor, producing an effect, for example, delaying the symptomatic progression of a cancer, symptoms thereof, or overcoming resistance development or reversing the resistance acquired due to pre-treatment, which is greater than the simple addition of the effects of each therapeutic agent administered by themselves. A synergistic effect can be calculated, for example, using suitable methods such as the Sigmoid-Emax equation (Holford, N. H. G. and Scheiner, L. B., Clin. Pharmacokinet. 6: 429-453 (1981)), the equation of Loewe additivity (Loewe, S. and Muischnek, H., Arch. Exp. Pathol Pharmacol. 114: 313-326 (1926)) and the median-effect equation (Chou, T. C. and Talalay, P., Adv. Enzyme Regul. 22: 27-55 (1984)). Each equation referred to above can be applied to experimental data to generate a corresponding graph to aid in assessing the effects of the drug combination. The corresponding graphs associated with the equations referred to above are the concentration-effect curve, isobologram curve and combination index curve, respectively. Synergy may be further shown by calculating the synergy score of the combination according to methods known by one of ordinary skill.

The term “pharmaceutically acceptable salts” refers to salts that retain the biological effectiveness and properties of the compound and which typically are not biologically or otherwise undesirable. The compound may be capable of forming acid addition salts by virtue of the presence of an amino group.

The terms “a” and “an” and “the” and similar references in the context of describing the invention (especially in the context of the following claims) are to be construed to cover both the singular and the plural, unless otherwise indicated herein or clearly contradicted by context. Where the plural form is used for compounds, salts, and the like, this is taken to mean also a single compound, salt, or the like.

COMPOUND A and its synthesis are specifically described in WO 2013/184757 as Example 5. COMPOUND A is an EGFR mutant specific inhibitor that is less effective against wild type EGFR. COMPOUND A recognizes mutant forms of EGFR, e.g. G719S, G719C, G719A, L858R, L861Q, exon 19 deletion, exon 20 insertion, T790M, T854A or D761Y mutant forms. The term EGFR inhibitor, also referred to as EGFR tyrosine kinase inhibitor, is defined herein to refer to a compound which binds to EGFR and/or inhibits or decreases EGFR kinase activity.

In one embodiment, COMPOUND A is in mesylate form. The preparation of the mesylate form of COMPOUND A is as follows:
(a) Crystalline Mesylate form B (mesylate trihydrate form) of COMPOUND A:
COMPOUND A (1.0 g) was dissolved in acetone (30 mL) by heating to 55° C. to form a solution. Methanesulfonic acid (325 μL) was added to acetone (50 mL), and the methanesulfonic acid/acetone (22.2 mL) was added to the COMPOUND A/acetone solution at a rate of 0.05 ml/min. Following precipitation, the resulting suspension was cooled to room temperature at a rate of 0.5° C./min, and crystals were collected by filtration, and dried for 4 hours at 40° C. under vacuum. The collected crystals (300 mg) were suspended in acetone/H₂O (6 mL; v/v=95/5) by heating to 50° C. The suspension was kept slurrying for 16 hours, and cooled to room temperature at 0.5° C./min. The crystals were collected by filtration and dried for 4 hours at 40° C. under vacuum. The structure of (R,E)-N-(7-chloro-1-(1-(4-(dimethylamino)but-2-enoyl)azepan-3-yl)-1H-benzo[d]imidazol-2-yl)-2-methylisonicotinamide (COMPOUND A) mesylate was confirmed by Differential Scanning calorimetry, X-Ray Powder Diffraction, and Elemental Analyses. Melting point (170.1° C.). Theoretical calculated: % C (54.8); % H (5.9); % N (14.2); % O (13.5); % S (5.4); and % Cl (6.0); C:N ratio: 3.86. Found: % C (52.0); % H (5.8); % N (13.3); % Cl (5.9); C:N ratio: 3.91. Stoichiometry: 1.01. In addition, crystalline mesylate form B of COMPOUND A, was prepared by suspending 300 mg of crystalline mesylate form A in 6 mL of acetone/H₂O (v/v=95/5) by heating to 50° C. The suspension was kept slurrying for 16 hours, and then the suspension was allowed to cool to room temperature at a rate of 0.5° C./min. The crystals were collected by filtration and afterwards dried for 4 hours at 40° C. under vacuum.
(b) Crystalline Mesylate form A (mesylate monohydrate form) of COMPOUND A 5.0 mL of dried acetone and 800 mg of mesylate form B (mesylate trihydrate Form) as obtained in (a) were added into a glass vial. The suspension was heated to 55° C. for 5 hours. DSC was checked to see if the transformation was complete.
Another 800 mg of the mesylate form B was converted to mesylate form A with the same method, the only difference was that the suspension was allowed to equilibrate at 20° C. (the ambient temperature in the lab), overnight.
In addition, crystalline mesylate form A was prepared by dissolving 1.0 g of free form of COMPOUND A in 30 mL of acetone by heating to 55° C. 325 μL, of methansulfonic acid was added to 50 mL of acetone and then 22.2 mL of methansulfonic acid acetone was added to free form solution at a rate of 0.05 ml/min. A precipitate was formed during the addition of methansulfonic acid, and the suspension was allowed to cool to room temperature at a rate of 0.5° C./min. The crystals were collected by filtration and afterwards dried for 4 hours at 40° C. under vacuum.

In another embodiment, COMPOUND A is in a form of hydrochloride salt. The preparation of COMPOUND A in a form of hydrochloride salt is described as follows:
1.0 g of amorphous form or free form of the COMPOUND A was dissolved in 50 mL of acetone by heating to 55° C. 22.2 mL of hydrochloride acid in acetone (0.1 mol/L) was added to free form solution at a rate of 0.05 ml/min. A precipitate was formed during the addition of hydrochloride acid, and the suspension was allowed to cool to room temperature at a rate of 0.5° C./min. The crystals were collected by filtration and afterwards dried for 4 hours at 40° C. under vacuum.
Another method of preparing the hydrochloride salt of COMPOUND A comprises, 850 mg of amorphous form or free form of the COMPOUND A were weighed out in a 20 ml vial. 4.25 ml of Acetonitrile was added to completely dissolve the compound. To this solution 6.86 ml of 0.6 N HCl were slowly added while stirring the solution. The solution turned yellow and solids precipitated out after 15 mins. The solution was stirred for 15 mins and then let to stand without stirring overnight. The solution was filtered and dried under vacuum at 40° C. for 8 hrs. A yellow solid was obtained as the final product.
The COMBINATION OF THE INVENTION further comprises a FGFR inhibitor.

The term FGFR inhibitor, also referred to as FGFR tyrosine kinase inhibitor, is defined herein to refer to a compound which binds to FGFR and/or inhibits or decreases the activity of one or more fibroblast growth factor receptors (e.g., FGFR1, FGFR2, FGFR3, FGFR4, or FGFR6).

In one embodiment, FGFR inhibitor of the COMBINATION OF THE INVENTION is a compound of formula II

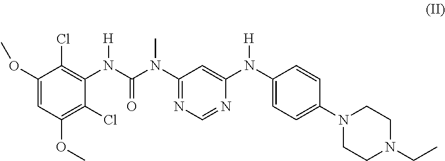 [SMILES: CCN1CCN(c2ccc(Nc3cc(N(C)C(=O)Nc4c(Cl)c(OC)cc(OC)c4Cl)ncn3)cc2)CC1]

3-(2,6-dichloro-3,5-dimethoxy-phenyl)-1-{6-[4-(4-ethyl-piperazin-1-yl)-phenylamino]-pyrimidin-4-yl}-1-methyl-urea (herein referred to as “COMPOUND B”), or a pharmaceutically acceptable salt thereof. COMPOUND B and its preparation are described in Example 145 of WO2006/000420.

Pharmaceutically acceptable salts of COMPOUND B are in particular a monophosphoric acid salt, or the hydrochloride salt, including dihydrate of the hydrochloride salt, and are disclosed in WO2011/071821.

Thus, in one embodiment, a FGFR inhibitor of the COMBINATION OF THE INVENTION is COMPOUND B in the form of a monophosphoric acid salt (3-(2,6-Dichloro-3,5-dimethoxy-phenyl)-1-{6-[4-(4-ethyl-piperazin-1-yl)-phenylamino]-pyrimid-4-yl}-1-methyl-urea monophosphate).

In another embodiment, a FGFR inhibitor of the COMBINATION OF THE INVENTION is COMPOUND B in the form of a hydrochloride salt. In a further embodiment, FGFR inhibitor of the COMBINATION OF THE INVENTION is COMPOUND B in the form of a hydrochloride salt, wherein the hydrochloride salt is a dihydrate.

While COMPOUND B is of particular interest, also other FGFR kinase inhibitors are possible. Examples of other suitable FGFR kinase inhibitors include, but are not limited to, the following compounds (including pharmaceutically acceptable salts or prodrugs thereof):
(a) dovitinib (known as TKI258 or previously CHIR258) is disclosed in WO02/22598 in example 109, as well as in Xin, X. et al., (2006), Clin. Cancer Res., Vol 12(16), p. 4908-4915; Trudel, S. et al., (2005), Blood, Vol. 105(7), p. 2941-2948). TKI258 has the following formula:

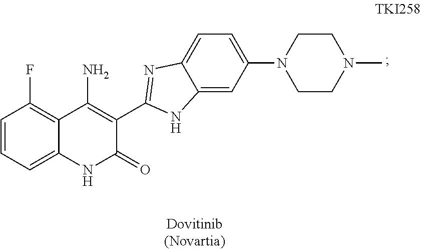 [SMILES: CN1CCN(c2ccc3nc(-c4c(N)c5c(F)cccc5[nH]c4=O)[nH]c3c2)CC1]

(b) AZD-4547 (AstraZeneca) which has the formula:

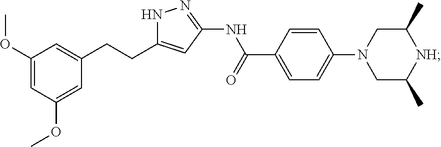 [SMILES: COc1cc(CCc2cc(NC(=O)c3ccc(N4C[C@@H](C)N[C@@H](C)C4)cc3)n[nH]2)cc(OC)c1]

(c) intedanib, brivanib (especially the alaninate), cediranib, masitinib, orantinib, ponatinib and E-7080;
(d) the following antibodies or related molecules:
HGS1036/FP-1039 (Human Genome Science/Five Prime) (see also J. Clin. Oncol. 28:15s, 2010, which is hereby incorporated by reference): soluble fusion protein consisting of the extracellular domains of human FGFR1 linked to the Fc region of human Immunoglobulin G1 (IgG1), designed to sequester and bind multiple FGF ligands and lock activation of multiple FGF receptors; MFGR1877S (Genentech/Roche): monoclonal antibody; AV-370 (AVEO): humanized antibody; GP369/AV-396b (AVEO): FGFR-IIIb-specific antibody; and HuGAL-FR21 (Galaxy Biotech): monoclonal antibody (FGFR2).

The structure of the active agents identified by code nos., generic or trade names may be taken from the actual edition of the standard compendium “The Merck Index” or from databases, e.g., Patents International (e.g., IMS World Publications). The corresponding content thereof is hereby incorporated by reference.

In one embodiment, FGFR inhibitor of the COMBINATION OF THE INVENTION selected from the group consisting of TKI258, or a pharmaceutically acceptable salt thereof; and 3-(2,6-dichloro-3,5-dimethoxy-phenyl)-1-{6-[4-(4-ethylpiperazin-1-1)-phenylamino]-pyrimidin-4-yl}-1-methyl-urea, or a pharmaceutically acceptable salt thereof, and AZD4547, or a pharmaceutically acceptable salt thereof.

Unless otherwise specified, or clearly indicated by the text, or not applicable, reference to therapeutic agents useful in the COMBINATION OF THE INVENTION includes both the free base of the compounds, and all pharmaceutically acceptable salts of the compounds.

In one aspect, the present invention relates to the COMBINATION OF THE INVENTION for simultaneous, separate or sequential use.

In one aspect, the present invention relates to the COMBINATION OF THE INVENTION for use in the treatment of a cancer.

The term “treating” or “treatment” is defined herein to refer to a treatment relieving, reducing or alleviating at least one symptom in a subject or affecting a delay of progression of a disease. For example, treatment can be the diminishment of one or several symptoms of a disease or complete eradication of a disease, such as cancer. Within the meaning of the present invention, the term “treat” also denotes to arrest, delay the progression and/or reduce the risk of developing resistance towards EGFR inhibitor treatment or otherwise worsening a disease.

The term “subject” or “patient” as used herein includes animals, which are capable of suffering from or afflicted with a cancer. Examples of subjects include mammals, e.g., humans, dogs, horses, cats, mice, rats and transgenic non-human animals. In the preferred embodiment, the subject is a human, e.g., a human suffering from a cancer, preferably lung cancer.

In one embodiment, the present invention relates to the COMBINATION OF THE INVENTION for use in the treatment of a lung cancer.

In a further embodiment, the present invention relates to the COMBINATION OF THE INVENTION for use in the treatment of a non-small cell lung cancer (NSCLC). The most common types of NSCLC are squamous cell carcinoma, large cell carcinoma, and lung adenocarcinoma. Less common types of NSCLC include pleomorphic, carcinoid tumor, salivary gland sarcoma, and unclassified sarcoma. In a more specific embodiment, the present invention relates to the COMBINATION OF THE INVENTION for use in the treatment of lung adenocarcinoma.

In one embodiment, the present invention relates to the COMBINATION OF THE INVENTION for use in the treatment of a cancer, particularly lung cancer, particularly non-small cell lung cancer (NSCLC), particularly lung adenocarcinoma, characterized by aberrant activation of EGFR, in particular amplification of EGFR, or somatic mutation of EGFR.

In a further embodiment, the present invention relates to the COMBINATION OF THE INVENTION for use in the treatment of a cancer, particularly lung cancer, particularly non-small cell lung cancer (NSCLC), particularly lung adenocarcinoma, characterized by harboring EGFR G719S mutation, EGFR G719C mutation, EGFR G719A mutation, EGFR L858R mutation, EGFR L861Q mutation, an EGFR exon 19 deletion, an EGFR exon 20 insertion, EGFR T790M mutation, EGFR T854A mutation or EGFR D761Y mutation, or any combination thereof.

In one embodiment, the present invention relates to the COMBINATION OF THE INVENTION for use in the treatment of a cancer, particularly lung cancer, particularly non-small cell lung cancer (NSCLC), particularly lung adenocarcinoma, characterized by harboring EGFR T790M mutation.

In one embodiment, EGFR T790M mutation is a de novo mutation. The term “de novo mutation” is defined herein to refer to an alteration in a gene that is detectable or detected in a subject, e.g., a mammal or human, before the onset of any treatment with an EGFR inhibitor. De novo mutation is a mutation which normally has occurred due to an error in the copying of genetic material or an error in cell division, e.g., de novo mutation may result from a mutation in a germ cell (egg or sperm) of one of the parents or in the fertilized egg itself, or from a mutation occurring in a somatic cell.

In another embodiment, EGFR T790M mutation is an acquired mutation, e.g., a mutation that is not detectable or detected before the cancer treatment but become detectable or detected in the course of the cancer treatment, particularly treatment with one or more EGFR inhibitors, e.g., gefitinib, erlotinib, or afatinib.

In one embodiment, the present invention relates to the COMBINATION OF THE INVENTION for use in the treatment of a cancer, particularly lung cancer, particularly non-small cell lung cancer (NSCLC), particularly lung adenocarcinoma, characterized by harboring EGFR T790M mutation in combination with any other mutation selected from the list comprising EGFR G719S mutation, EGFR G719C mutation, EGFR G719A mutation, EGFR L858R mutation, EGFR L861Q mutation, an EGFR exon 19 deletion, and an EGFR exon 20 insertion.

In one embodiment, the present invention relates to the COMBINATION OF THE INVENTION for use in the treatment of a cancer, particularly lung cancer, particularly non-small cell lung cancer (NSCLC), particularly lung adenocarcinoma, characterized by harboring EGFR T790M mutation in combination with any other mutation selected from the list consisting of EGFR G719S mutation, EGFR G719C mutation, EGFR G719A mutation, EGFR L858R mutation, EGFR L861Q mutation, an EGFR exon 19 deletion, and an EGFR exon 20 insertion, wherein EGFR T790M mutation is a de novo mutation.

In another embodiment, the present invention relates to the COMBINATION OF THE INVENTION for use in the treatment of a cancer, particularly lung cancer, particularly non-small cell lung cancer (NSCLC), particularly lung adenocarcinoma, characterized by harboring EGFR T790M mutation in combination with any other mutation selected from the list consisting of EGFR G719S mutation, EGFR G719C mutation, EGFR G719A mutation, EGFR L858R mutation, EGFR L861Q mutation, an EGFR exon 19 deletion, and an EGFR exon 20 insertion, wherein EGFR T790M mutation is an acquired mutation.

In one embodiment, the present invention relates to the COMBINATION OF THE INVENTION for use in the treatment of a cancer, particularly lung cancer, particularly non-small cell lung cancer (NSCLC), particularly lung adenocarcinoma, characterized by harboring EGFR mutation selected from the group consisting of G719S, G719C, G719A, L858R, L861Q, an exon 19 deletion mutation, and an exon 20 insertion mutation. In a preferred embodiment, the present invention relates to the COMBINATION OF THE INVENTION for use in the treatment of a cancer characterized by harboring at least one of the following mutations: EGFR L858R and an EGFR exon 19 deletion.

In one embodiment, the present invention relates to the COMBINATION OF THE INVENTION for use in the treatment of a cancer, particularly lung cancer, particularly non-small cell lung cancer (NSCLC), particularly lung adenocarcinoma, characterized by harboring EGFR mutation selected from the group consisting of G719S, G719C, G719A, L858R, L861Q, an exon 19 deletion mutation, and an exon 20 insertion mutation, and further characterized by harboring at least one further EGFR mutation selected from the group consisting of T790M, T854A and D761Y mutation.

In a preferred embodiment, the present invention relates to the COMBINATION OF THE INVENTION for use in the treatment of a cancer, particularly lung cancer, particularly non-small cell lung cancer (NSCLC), particularly lung adenocarcinoma, characterized by harboring EGFR L858R mutation or EGFR exon 19 deletion, and further harboring an EGFR T790M mutation.

In one embodiment, the present invention relates to the COMBINATION OF THE INVENTION for use in the treatment of a cancer, particularly lung cancer, particularly non-small cell lung cancer (NSCLC), particularly lung adenocarcinoma, wherein the cancer is resistant to a treatment with an EGFR tyrosine kinase inhibitor, or is developing a resistance to a treatment with an EGFR tyrosine kinase inhibitor, or is under high risk of developing a resistance to a treatment with an EGFR tyrosine kinase inhibitor.

In another embodiment, the present invention relates to the COMBINATION OF THE INVENTION for use in the treatment of a cancer, particularly lung cancer, particularly non-small cell lung cancer (NSCLC), particularly lung adenocarcinoma, wherein the cancer is resistant to a treatment with an EGFR tyrosine kinase inhibitor, or is developing a resistance to a treatment with an EGFR tyrosine kinase inhibitor, or is under high risk of developing a resistance to a treatment with an EGFR tyrosine kinase inhibitor, wherein the EGFR tyrosine kinase inhibitor is selected from the group consisting of erlotinib, gefitinib and afatinib.

The COMBINATION OF THE INVENTION is also suitable for the treatment of poor prognosis patients, especially such poor prognosis patients having a cancer, particularly lung cancer, particularly non-small cell lung cancer (NSCLC), particularly lung adenocarcinoma, which becomes resistant to treatment employing an EGFR inhibitor, e.g. a cancer of such patients who initially had responded to treatment with an EGFR inhibitor and then relapsed. In a further example, said patient has not received treatment employing a FGFR inhibitor. This cancer may have acquired resistance during prior treatment with one or more EGFR inhibitors. For example, the EGFR targeted therapy may comprise treatment with gefitinib, erlotinib, lapatinib, XL-647, HKI-272 (Neratinib), BIBW2992 (Afatinib), EKB-569 (Pelitinib), AV-412, canertinib, PF00299804, BMS 690514, HM781-36b, WZ4002, AP-26113, cetuximab, panitumumab, matuzumab, trastuzumab, pertuzumab, COMPOUND A of the present invention, or a pharmaceutically acceptable salt thereof. In particular, the EGFR targeted therapy may comprise treatment with gefitinib, erlotinib, and afatinib. The mechanisms of acquired resistance include, but are not limited to, developing a second mutation in the EGFR gene itself, e.g. T790M, EGFR amplification; and/or FGFR deregulation, FGFR mutation, FGFR ligand mutation, FGFR amplification, or FGFR ligand amplification. In one embodiment, the acquired resistance is characterized by the presence of T790M mutation in EGFR.

In another embodiment, the COMBINATION OF THE INVENTION is also suitable for the treatment of patients having a cancer, particularly lung cancer, particularly non-small cell lung cancer (NSCLC), particularly lung adenocarcinoma, wherein the cancer is developing resistance to treatment employing an EGFR inhibitor as a sole therapeutic agent.

In another embodiment, the COMBINATION OF THE INVENTION is also suitable for the treatment of patients having a cancer, particularly lung cancer, particularly non-small cell lung cancer (NSCLC), particularly lung adenocarcinoma, wherein the cancer is under a high risk of developing a resistance to a treatment with an EGFR inhibitor as a sole therapeutic agent. Since almost all cancer patients harboring EGFR mutations, in particular NSCLC patients, develop with the time resistance to the treatment with such EGFR tyrosine kinase inhibitors as gefitinib, erlotinib, or afatinib, a cancer of said patient is always under a high risk of developing a resistance to a treatment with an EGFR inhibitor as a sole therapeutic agent. And thus, cancers harboring EGFR G719S mutation, EGFR G719C mutation, EGFR G719A mutation, EGFR L858R mutation, EGFR L861Q mutation, an EGFR exon 19 deletion, an EGFR exon 20 insertion, EGFR T790M mutation, EGFR T854A mutation or EGFR D761Y mutation, or any combination thereof are under a high risk of developing a resistance to a treatment with an EGFR inhibitor as a sole therapeutic agent.

In a further embodiment, the present invention relates to the COMBINATION OF THE INVENTION for use in the treatment of a cancer, particularly lung cancer, particularly non-small cell lung cancer (NSCLC), particularly lung adenocarcinoma, further characterized by aberrant activation of FGFR, in particular amplification of FGFR, or somatic mutation of FGFR, or aberrant expression of a FGF ligand, e.g., gene amplification of FGFR1, FGFR2, FGFR3 or FGFR4; translocation and fusion of FGFR1 to other genes; or somatic activating mutations of FGFR1, FGFR2, FGFR3 or FGFR4, or aberrant expression of a FGF ligand.

In another aspect, the present invention relates to the pharmaceutical composition comprising the COMBINATION OF THE INVENTION and at least one pharmaceutically acceptable carrier.

As used herein, the term “pharmaceutically acceptable carrier” includes generally recognized as safe (GRAS) solvents, dispersion media, coatings, surfactants, antioxidants, preservatives (e.g., antibacterial agents, antifungal agents), isotonic agents, absorption delaying agents, salts, preservatives, drug stabilizers, binders, excipients, disintegration agents, lubricants, sweetening agents, flavoring agents, dyes, buffering agents (e.g., maleic acid, tartaric acid, lactic acid, citric acid, acetic acid, sodium bicarbonate, sodium phosphate, and the like), and the like and combinations thereof, as would be known to those skilled in the art (see, for example, Remington's Pharmaceutical Sciences, 18th Ed. Mack Printing Company, 1990, pp. 1289-1329). Except insofar as any conventional carrier is incompatible with COMPOUND A or COMPOUND B its use in the pharmaceutical compositions or medicaments is contemplated.

In another aspect, the present invention relates to use of COMPOUND A or any pharmaceutical acceptable salt thereof for the preparation of a medicament for use in combination with an FGFR inhibitor for the treatment of lung cancer. In another aspect, the present invention relates to use of the FGFR inhibitor for the preparation of a medicament for use in combination with COMPOUND A or any pharmaceutical acceptable salt thereof for the treatment of lung cancer, particularly non-small cell lung cancer (NSCLC), particularly lung adenocarcinoma.

In another aspect, the present invention relates to a method of treating a lung cancer, particularly non-small cell lung cancer (NSCLC), particularly lung adenocarcinoma, comprising simultaneously, separately or sequentially administering to a subject in need thereof the COMBINATION OF THE INVENTION in a quantity which is jointly therapeutically effective against said lung cancer.

The individual therapeutic agents of the COMBINATION OF THE INVENTION may be administered separately at different times during the course of therapy or concurrently in divided or single combination forms. For example, the method of treating a cancer, particularly lung cancer, particularly non-small cell lung cancer (NSCLC), particularly lung adenocarcinoma, according to the invention may comprise: (i) administration of COMPOUND A in free or pharmaceutically acceptable salt form, and (ii) administration of a FGFR inhibitor, preferably COMPOUND B, in free or pharmaceutically acceptable salt form, simultaneously or sequentially in any order, in jointly therapeutically effective amounts, preferably in synergistically effective amounts, e.g. in daily or intermittently dosages corresponding to the amounts described herein.

The term “administration” is also intended to include treatment regimens in which the therapeutic agents are not necessarily administered by the same route of administration or at the same time.

In one embodiment, COMPOUND A and COMPOUND B are administered for the first 21 days of every 28 day cycle and then just COMPOUND A for the last 7 days of every 28 day cycle. In another embodiment, COMPOUND A is administered daily alone during the first cycle, and together with COMPOUND B starting from the second cycle, where starting from the second cycle COMPOUND A and COMPOUND B are administered for the first 21 days of every 28 day cycle and then just COMPOUND A for the last 7 days of every 28 day cycle.

The term “jointly therapeutically active” or “joint therapeutic effect” as used herein means that the therapeutic agents may be given separately (in a chronologically staggered manner, especially a sequence-specific manner) in such time intervals that they prefer, in mammal, especially human, to be treated, still show a beneficial (preferably synergistic) interaction (joint therapeutic effect). Whether this is the case can, inter alia, be determined by following the blood levels, showing that both therapeutic agents are present in the blood of the human to be treated at least during certain time intervals.

It can be shown by established test models that a COMBINATION OF THE INVENTION results in the beneficial effects described herein before. The person skilled in the art is fully enabled to select a relevant test model to prove such beneficial effects. The pharmacological activity of a COMBINATION OF THE INVENTION may, for example, be demonstrated in a clinical study or in an in vivo or in vitro test procedure as essentially described hereinafter.

The term “effective amount” or “therapeutically effective amount” of a combination of therapeutic agents is defined herein to refer to an amount sufficient to provide an observable improvement over the baseline clinically observable signs and symptoms of the cancer treated with the combination.

In certain embodiments, a therapeutic amount or dose of COMPOUND A may range from about 0.1 mg/kg to about 500 mg/kg, alternatively from about 1 to about 50 mg/kg. In general, treatment regimens comprise administration to a patient in need of such treatment from about 10 mg to about 1000 mg of COMPOUND A per day in single or multiple doses (such as two, three, or four times daily). Therapeutic amounts or doses will also vary depending on route of administration, as well as the possibility of co-usage with other agents.

In one embodiment, COMPOUND A is administered orally in a daily dosage in the range of 50 mg to 300 mg, in particular in a daily dosage of 50 mg, 75 mg, 100 mg, 150 mg, 200 mg, or 300 mg. In a preferred embodiment, COMPOUND A is administered orally in a daily dosage of 75 mg.

In one embodiment, COMPOUND B is administered orally in a daily dosage in the range of 25 mg to 200 mg, particularly in the range of 50 mg to 150 mg, particularly in the range of 75 mg to 125 mg. Particularly COMPOUND B is administered in the dose of 50 mg, 75 mg, 100 mg, or 125 mg. In a preferred embodiment, COMPOUND B is administered orally in a daily dosage of 75 mg. In another preferred embodiment, COMPOUND B is administered orally in a daily dosage of 125 mg.

It is understood that each therapeutic agent may be conveniently administered, for example, in one individual dosage unit or divided into multiple dosage units. It is further understood that that each therapeutic agent may be conveniently administered in doses once daily or doses up to four times a day.

In one embodiment, the present invention relates to a commercial package comprising the COMBINATION OF THE INVENTION and instructions for simultaneous, separate or sequential administration of the COMBINATION OF THE INVENTION to a patient in need thereof. In one embodiment, the present invention provides a commercial package comprising the EGFR inhibitor COMPOUND A, or a pharmaceutically acceptable salt thereof, and instructions for the simultaneous, separate or sequential use with a FGFR inhibitor, preferably COMPOUND B or a pharmaceutically acceptable salt thereof, for use in the treatment of a cancer, particularly lung cancer, particularly non-small cell lung cancer (NSCLC), particularly lung adenocarcinoma, and preferably wherein the cancer is characterized by a mutant EGFR; for example, wherein the mutant EGFR comprises G719S, G719C, G719A, L858R, L861Q, an exon 19 deletion mutation, an exon 20 insertion mutation, EGFR T790M, T854A or D761Y mutation, or any combination thereof, and preferably wherein said cancer has acquired resistance during prior treatment with one or more EGFR inhibitors or developing a resistance to a treatment with one or more EGFR inhibitors, or under high risk of developing a resistance to a treatment with an EGFR inhibitor.

The following Examples illustrate the invention described above, but are not, however, intended to limit the scope of the invention in any way. Other test models known as such to the person skilled in the pertinent art can also determine the beneficial effects of the claimed invention.

### EXAMPLES

### Example 1: Dose-Dependent Growth Inhibition

Percent growth inhibition was identified for the NCI-H1975 EGFR mutant cell line following 72 hours of treatment with several doses of (FIG. 1):
(a) COMPOUND A as a single agent,
(b) COMPOUND A plus FGF2, or
(c) COMPOUND A plus both FGF2 and COMPOUND B (500 nM).

Methods: Proliferation Assay Methods (FIG. 1):

The human non-small cell lung cancer (NSCLC) cell line, NCI-H1975 harboring the EGFR mutations L858R and T790M was purchased from American Type Culture Collection (ATCC). The cells were cultured in RPMI-1640 growth medium (ATCC, catalog number 20-2001), supplemented with 10% fetal bovine serum (GIBCO, catalog number F4135) at 37° C. in a humidified 5% CO 2 incubator. For the cell proliferation assay, NCI-H1975 cells were seeded at a density of 3000 cells per well in a tissue cultured treated 96-well plate (Corning, catalog number 3904) and allowed to attach overnight.

The cells were then exposed to the following drug treatments:
(a) dose response of COMPOUND A,
(b) dose response of COMPOUND A and 50 ng/ml exogenous FGF2 ligand (R&D Systems, catalog number 233-FB-025),
(c) dose response of COMPOUND A, 50 ng/ml exogenous FGF2 ligand and COMPOUND B.

After 72 hours of treatment, cell growth was analyzed by measuring ATP levels using the CellTiter Glo assay (Promega, catalog number G7572) as recommended by the manufacturer's instructions. IC50 values were calculated using GraphPad Prism software.

Results:

FIG. 1 illustrates three findings: (1) that COMPOUND A inhibits growth of the NCI-H1975 EGFR mutant (L858R, T790M) cell line in a dose dependent fashion, (2) that addition of recombinant fibroblast growth factor 2 (FGF2) prevents growth inhibition by COMPOUND A, and (3) that growth inhibition is restored by adding COMPOUND B in addition to both COMPOUND A plus FGF2. These findings indicate that activation of FGFR signaling can rescue tumor cells from inhibition of mutant EGFR-mediated growth signaling and that combining COMPOUND B treatment with COMPOUND A can sensitize tumor cells to inhibition of mutant EGFR in contexts where FGFR signaling is activated.

### Example 2: Activation of the FGFR Pathway Following EGFR Inhibition

Activation of the FGFR pathway following EGFR inhibition in an EGFR mutant non-small cell lung cancer (NSCLC) cell line was observed (FIG. 2).

Method: pEGFR3 and tEGFR3 Assay

The following kits were used to assay pEGFR3 and tEGFR3: Human Phospho-FGFR3 (pFGFR3) (R&D Systems, Catalog number DYC2719-2), and Human Total FGFR3 (tFGFR3) (R&D Systems, Catalog number DYC766-2).

The NCI-H1975 cells were plated at a density 20,000 cells per well in 6-well plates and allowed to attach overnight at 37° C. in a humidified 5% CO₂ incubator. Cells were then exposed to the following drug treatments:
(a) 500 nM COMPOUND B,
(b) 30 nM COMPOUND A,
(c) 500 nM COMPOUND B and 30 nM COMPOUND A combination.

Cells were treated for 2, 4, 6, 24, 48, 72, 96, 120, 144, 168 hours. After treatment, cells were washed twice with cold PBS and solubilized in MSD Tris Lysis Buffer (catalog number R60TX-2) with MSD Inhibitor Pack (Phosphatase and Protease Inhibitor Cocktails, (catalog number R70AA-1) according to the manufacturer's instructions. Lysates were then assayed for both phosphoFGFR3 and total FGFR3 according to the manufacturer's instructions.

Method: pERK and pEGFR Assay

The following kits were used to assay pERK and pEGFR: AlphaScreen SureFire ERK1/2 (Thr202/Tyr204) Assay Kit (Perkin Elmer, catalog number TGRES500) and AlphaScreen SureFire EGFR (Tyr1068) Assay Kit (Perkin Elmer, catalog number TGRERS500).

The NCI-H1975 cells were plated at a density 20,000 cells per well in 6-well plates and allowed to attach overnight at 37° C. in a humidified 5% CO₂ incubator. Cells were then exposed to the following drug treatments:
(a) 500 nM COMPOUND B,
(b) 30 nM COMPOUND A,
(c) 500 nM COMPOUND B and 30 nM COMPOUND A combination.

Cells were treated for 2, 4, 6, 24, 48, 72, 96, 120, 144, 168 hours. After treatment, medium was aspirated off and 1× Lysis Buffer provided in the kit was added to cells. Lysates were then assayed for pERK (p-Thr202/Tyr204) and pEGFR (Tyr1068) according to the manufacturer's instructions.

Results:

Fold-changes in pEGFR (A), tFGFR3 (B), pFGFR3 (C), and pERK (D) following treatment of the EGFR mutant NCI-H1975 cell line with COMPOUND B (500 nM) alone, COMPOUND A (30 nM) alone, or the combination are shown relative to vehicle treated control over a time-course (FIG. 2).

FIG. 2A illustrates that COMPOUND A (30 nM) effectively and durably inhibits mutant EGFR signaling, as assessed by phosphorylation of EGFR on residue Y1068 (pEGFR; indicative of pathway activation), in the NCI-H1975 cell line, while COMPOUND B has no effect on pEGFR.

FIG. 2B illustrates that COMPOUND A treatment increased total FGFR3 (tFGFR3) modestly (about 2-fold) only at the 120 and 144 hour (h) time points.

FIG. 2C illustrates that COMPOUND A treatment leads to increased phosphorylation of FGFR3, indicative of FGFR pathway activation on and after 120 hours of treatment, and that COMPOUND B co-treatment prevents this increase of pFGFR3 as well as pERK, indicative of MAPK pathway activation status (FIG. 2D).

Together, these data indicate that inhibition of mutant EGFR for 5 or more days leads to FGFR pathway activation and that co-treatment with COMPOUND B can inhibit the compensatory FGFR signaling.

### Example 3: Relative Cell Number Over Time

Further data on relative cell number following treatment of the EGFR mutant NCI-H1975 cell line over time shows that combining COMPOUND B with COMPOUND A resulted in further inhibition of growth of a cell line in vitro (FIG. 3).

Method: Real Time Cell Viability Assay Using the RTCA SP xCelligence Instrument:

NCI-H1975 cells were plated as 1000 cells per well in 140 ul medium in 96 well E-plates (catalog number 05232368001) specific to the xCelligence instrument. A protocol was created by completing the experiment layout and length of time for cell incubation according to the instrument operator's manual. Cells were allowed to adhere overnight at 37° C., 5% CO₂ in the xCelligence incubator. Cells were then exposed to achieve final concentration of the following treatments:
(a) DMSO (0.3% final),
(b) 30 nM COMPOUND A,
(c) 30 nM COMPOUND A and 500 nM COMPOUND B combination.

After compound addition, cells are placed in the Xcelligence incubator to read for at least 200 hours. After completion of treatment, stop and release plates from the instrument. Analyze growth results using the RTCA analyzer software.

Results:

It has been found that that COMPOUND A (30 nM) partially inhibits growth of the EGFR mutant NCI-H1975 cell line, and the combination activity was observed with COMPOUND B co-treatment (500 nM) (FIG. 3). Significantly, combination activity is only observed only after more than roughly 100 hours of co-treatment. This combination activity is coincident with FGFR pathway activation status illustrated in FIG. 2C.

### Example 4: In Vivo Primary, Patient-Derived, Tumor Xenograft Experiment

In a patient derived xenograft model, harboring the EGFR mutations, L858R and T790M, combination activity was observed in vivo with COMPOUND B and COMPOUND A co-treatment (FIG. 4).

Method:

Patient derived tumor fragments from stock mice inoculated with selected primary human NSCLC tissues were harvested and used for inoculation into BALB/c nude mice. Each mouse was inoculated subcutaneously at the right flank with primary human NSCLC model LU1868 (EGFR mutant L858R; T790M) fragment (2-4 mm in diameter) for tumor development. The treatment was started when the average tumor size reached about 150 mm³. On day 0, mice were allocated randomly into experimental groups according to their tumor sizes. Each group consisted of 8 mice.

Treatments were administrated to the mice orally from day 0 through day 20 for vehicle, COMPOUND B (15 mg/kg/day) single agent, and COMPOUND A (10 mg/kg/day) single agent groups; and to the group treated with COMPOUND B (15 mg/kg/day)+COMPOUND A (10 mg/kg/day) from day 0 through day 48. Tumor size was measured twice weekly in two dimensions using a caliper, and the volume is expressed in mm³ using the formula: V=0.5 a×b2, where a and b are the long and short diameters of the tumor, respectively.

Results:

It has been found that single agent COMPOUND B (15 mg/kg/day) had no effect on tumor growth; single agent COMPOUND A (10 mg/kg/day) was partially efficacious; and co-treatment with both COMPOUND A (10 mg/kg/day) and COMPOUND B (15 mg/kg/day) led to complete and durable tumor regression (FIG. 4). These data demonstrate that COMPOUND B can provide combination benefit with COMPOUND A co-treatment in a primary human tumor in vivo. Thus, these data support combining COMPOUND B with COMPOUND A treatment in EGFR mutant lung cancer, specifically in tumors with evidence of FGFR activation after initiation of COMPOUND A treatment.

### Example 5

This study is a phase Ib, multicenter, open-label dose escalation and expansion study of COMPOUND B (3-(2,6-Dichloro-3,5-dimethoxy-phenyl)-1-{6-[4-(4-ethyl-piperazin-1-yl)-phenylamino]-pyrimid-4-yl}-1-methyl-urea) in combination with COMPOUND A ((R,E)-N-(7-chloro-1-(1-(4-(dimethylamino)but-2-enoyl)azepan-3-yl)-1H-benzo[d]imidazol-2-yl)-2-methylisonicotinamide) in adult patients with unresectable EGFR T790M mutated non-small cell lung cancer (lung adenocarcinoma). This study is designed to test the hypothesis that formal acquired resistance can be inhibited and/or delayed if the adaptive response can be inhibited via the combination of COMPOUND A and COMPOUND B. This study is also designed to establish the dose of COMPOUND A that can safely be combined with up to 125 mg qd of COMPOUND B (Maximum Tolerated Dose, MTD).

Patient Population:

Patients, male and female≧18 years of age, must have advanced and unresectable NSCLC (lung adenocarcinoma) with an acquired T790M EGFR mutation. The positivity of T790M is defined by therascreen EGFR RGQ PCR kits (Qiagen).

The following patients are not eligible for the study: (i) previously treated with any approved EGFR inhibitor targeting EGFR T790M mutation (e.g., afatinib); (ii) previously treated with more than total of 3 previous anti-neoplastic therapies in the advanced setting; (iii) previously treated with more than one previous approved EGFR inhibitor; (iv) undergone previous treatment with any investigational agent known to inhibit EGFR (mutant or wild-type); (v) undergone prior or current treatment with a FGFR inhibitor (unless agreed on a case by case basis).

Study Design:

The MTD and/or RDE (Recommended dose for expansion) of the combination of oral COMPOUND B with oral COMPOUND A is determined in this multi-center, open-label phase Ib dose escalation trial. COMPOUND B and COMPOUND A will be given concurrently. Upon identification of the MTD or RDE, an expansion part will be opened to patient enrollment enrolled to an expansion arm for assessment the anti-tumor activity of the COMPOUND A in combination with COMPOUND B, in patients with EGFR-mutant NSCLC T790M.

The escalation part will enroll approximately≧21 patients with EGFR-T790M NSCLC. The dose escalation is guided by a Bayesian logistic regression model (BLRM) based on dose limiting toxicity (DLT) data of the combination and by two Bayesian linear regression model that relate the dose and the exposure of COMPOUND B and COMPOUND A, taking into account any interaction between the two drugs. Complementing the BLRM, the Bayesian modelling of the exposure of COMPOUND B and COMPOUND A will provide additional information that will allow efficient targeting of dose-combinations with desired exposure.

Approximately 40 patients with EGFR-T790M NSCLC will be enrolled to the expansion part. Patients will be treated for an initial one cycle (28-day “run-in”) of COMPOUND A and biopsies obtained at baseline and at the completion of at least 21 days of the 28-day cycle. With the initiation of Cycle 2 of COMPOUND A, COMPOUND B will also be administered. The 28-day “run-in” of COMPOUND A and the accompanying biopsies may be discontinued after a minimum of ten patients have underwent paired biopsies. Patients enrolled subsequent to the discontinuation of biopsies receive COMPOUND A and COMPOUND B concurrently throughout.

Treatment for both parts will be administered in 28-day cycles and the duration of the study is approximately 24 months (from FPFV to LPLV).

Dosing and Regiment:

COMPOUND A and COMPOUND B will be administered orally as flat-fixed doses and not by body weight or body surface area. The starting dose is 75 mg for each study drugs. Daily dose of COMPOUND A may also be 50 mg, 75 mg, 100 mg, 150 mg, 200 mg and 300 mg. Daily dose of COMPOUND B may also be 50 mg, 75 mg, 100 mg, and 125 mg. The dose of each drug will be adjusted based on the response of a patient. Table 1 describes the starting dose and the dose levels that may be evaluated during this trial.

Patients take both COMPOUND A and COMPOUND B for the first 21 days of every 28 day cycle (the drugs can be taken in any order) and then just COMPOUND A for the last 7 days of every 28 day cycle. In the dose-expansion phase, patients will be administered daily COMPOUND A alone during the first cycle and together with COMPOUND B starting from the second cycle and according to the regimen described above.

TABLE 1
Provisional dose levels

| | Proposed daily dose of | Proposed daily dose of |
|---|---|---|
| Dose level* | COMPOUND A* | COMPOUND B* |
| -1a | 50 mg | 75 mg |
| -1b | 75 mg | 50 mg |
| -1c | 50 mg | 50 mg |
| Starting dose level 1 | 75 mg | 75 mg |
| 2a | 150 mg | 75 mg |
| 2b (alternative to 2a) | 75 mg | 100 mg |
| 3a | 150 mg | 100 mg |
| 3b (alternative to 3a) | 75 mg | 125 mg |
| 4a | 150 mg | 125 mg |
| 4b (alternative to 4a) | 100 mg | 125 mg |
| 5a | 300 mg | 125 mg |
| 5b | 200 mg | 125 mg |

*It is possible for additional and/or intermediate dose levels to be added during the course of the study Cohorts may be added at any dose level below the MTD in order to better understand safety, PK and/or PD.

Treatment Duration:

Patients continue to be treated with study drugs until they experience unacceptable toxicities for which symptomatic management and/or dose reduction are not considered sufficient, disease progression, start of new anti-cancer therapy, withdrawal of consent, failure to comply with study requirements, and/or discretion of the investigator or sponsor.

Dose escalation will continue until identification of the MTD or a suitable lower dose for expansion.

Objectives and Related Endpoint:

The objectives of the study are (i) to estimate the MTD and/or RDE of the combination, which is measured by incidence of DLT; (ii) to estimate safety the combination, which is measured by incidence of adverse events and serious adverse events, including changes in hematology and chemistry values, vital signs and electrocardiograms; (iii) to estimate tolerability of the combination, which is measured by frequency of dose interruptions, reductions, and dose intensity; (iv) to evaluate the preliminary antitumor activity of COMPOUND B and COMPOUND A, which is measured by TTP, ORR, and 6 month PFS rate and other PFS measures using RECIST version 1.1; (v) to evaluate PK of COMPOUND B and COMPOUND A in the combination setting, measured by plasma concentration vs. time profiles and plasma PK parameters of COMPOUND B and COMPOUND A.

1. A pharmaceutical combination comprising (a) a compound of formula I 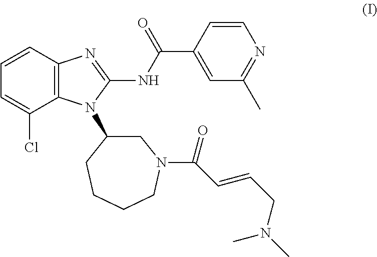 [SMILES: Cc1cc(C(=O)Nc2nc3cccc(Cl)c3n2[C@@H]2CCCCN(C(=O)/C=C/CN(C)C)C2)ccn1]
(R,E)-N-(7-chloro-1-(1-(4-(dimethylamino)but-2-enoyl)azepan-3-yl)-1H-benzo[d]imidazol-2-yl)-2-methylisonicotinamide, or a pharmaceutically acceptable salt thereof, and (b) a FGFR inhibitor.

2. The pharmaceutical combination according to claim 1, wherein the FGFR inhibitor is selected from the group consisting of 3-(2,6-dichloro-3,5-dimethoxy-phenyl)-1-{6-[4-(4-ethylpiperazin-1-1)-phenylamino]-pyrimidin-4-yl}-1-methyl-urea, or a pharmaceutically acceptable salt thereof, TK1258, or a pharmaceutically acceptable salt thereof; and AZD4547, or a pharmaceutically acceptable salt thereof.

3. The pharmaceutical combination according to claim 1, wherein the FGFR inhibitor is 3-(2,6-Dichloro-3,5-dimethoxy-phenyl)-1-{6-[4-(4-ethyl-piperazin-1-yl)-phenylamino]-pyrimid-4-yl}-1-methyl-urea, or a pharmaceutically acceptable salt thereof.

4. The pharmaceutical combination according to claim 3, wherein the FGFR inhibitor is 3-(2,6-Dichloro-3,5-dimethoxy-phenyl)-1-{6-[4-(4-ethyl-piperazin-1-yl)-phenylamino]-pyrimid-4-yl}-1-methyl-urea monophosphate.

5. The pharmaceutical combination according to claim 1, wherein (R,E)-N-(7-chloro-1-(1-(4-(dimethylamino)but-2-enoyl)azepan-3-yl)-1H-benzo[d]imidazol-2-yl)-2-methylisonicotinamide is in mesylate form.

6. The pharmaceutical combination according to claim 1, wherein (R,E)-N-(7-chloro-1-(1-(4-(dimethylamino)but-2-enoyl)azepan-3-yl)-1H-benzo[d]imidazol-2-yl)-2-methylisonicotinamide is in the form of hydrochloride salt.

7. The pharmaceutical combination according to claim 1 for simultaneous, separate or sequential use.

8. The pharmaceutical combination according to claim 1 for use in the treatment of a cancer.

9. The pharmaceutical combination according to claim 8, wherein the cancer is a lung cancer.

10. The pharmaceutical combination according to claim 9, wherein the lung cancer is a non-small cell lung cancer, in particular lung adenocarcinoma.

11. The pharmaceutical combination according to claim 8, wherein the cancer characterized by aberrant activation of EGFR, in particular amplification of EGFR, or somatic mutation of EGFR.

12. The pharmaceutical combination according to claim 8, wherein the cancer is characterized by harboring EGFR G719S mutation, EGFR G719C mutation, EGFR G719A mutation, EGFR L858R mutation, EGFR L861Q mutation, an EGFR exon 19 deletion, an EGFR exon 20 insertion, EGFR T790M mutation, EGFR T854A mutation or EGFR D761Y mutation, or any combination thereof.

13. The pharmaceutical combination according to claim 8, wherein the cancer is characterized by harboring EGFR T790M mutation.

14. The pharmaceutical combination of claim 8, wherein the cancer is characterized by harboring EGFR mutation selected from the group consisting of G719S, G719C, G719A, L858R, L861Q, an exon 19 deletion mutation, and an exon 20 insertion mutation, or any combination thereof.

15. The pharmaceutical combination of claim 14, wherein the cancer is characterized by harboring at least one further EGFR mutation selected from the group consisting of T790M, T854A and D761Y mutation.

16. The pharmaceutical combination according to claim 14, wherein the cancer is characterized by harboring a further EGFR T790M mutation.

17. The pharmaceutical combination according to claim 8, wherein the cancer is resistant to a treatment with an EGFR tyrosine kinase inhibitor, or developing a resistance to a treatment with an EGFR tyrosine kinase inhibitor, or under high risk of developing a resistance to a treatment with an EGFR tyrosine kinase inhibitor.

18. The pharmaceutical combination according to claim 17, wherein the EGFR tyrosine kinase inhibitor is selected from the group consisting of erlotinib, gefitinib and afatinib.

19. A pharmaceutical composition comprising the pharmaceutical combination according to claim 1 and at least one pharmaceutically acceptable carrier.

20. Use of compound of formula I or any pharmaceutical acceptable salt thereof for the preparation of a medicament for use in combination with an FGFR inhibitor according to claim 2 for the treatment of lung cancer.

21. Use of the FGFR inhibitor according to claim 2 for the preparation of a medicament for use in combination with compound of formula I or any pharmaceutical acceptable salt thereof for the treatment of lung cancer.

22. A method of treating lung cancer comprising simultaneously, separately or sequentially administering to a subject in need thereof the pharmaceutical combination according to claim 1 in a quantity which is jointly therapeutically effective against said lung cancer.

23. A commercial package for use in the treatment of lung cancer comprising the pharmaceutical combination according to claim 1 and instructions for simultaneous, separate or sequential administration of said pharmaceutical combination to a patient in need thereof.
